(19) United States
(12) Patent Application Publication
(10) Pub. No.: US 2013/0102076 A1
(43) Pub. Date: Apr. 25, 2013

(54) Systems and methods for growing photosynthetic organisms

(71) Applicants: Jason D. Licamele, Scottsdale, AZ (US); Thomas J. Kulaga, Chandler, AZ (US)

(72) Inventors: Jason D. Licamele, Scottsdale, AZ (US); Thomas J. Kulaga, Chandler, AZ (US)

(21) Appl. No.: 13/694,052

(22) Filed: Oct. 24, 2012

Related U.S. Application Data
(60) Provisional application No. 61/550,912, filed on Oct. 24, 2011.
(60) Provisional application No. 61/578,107, filed on Dec. 20, 2011.

(51) Int. Cl. C12M 3/00; C12N 1/12; C12N 5/04

(52) U.S. Cl. 435/420; 435/257.1; 435/292.1

(57) ABSTRACT

Methods and apparatus for promoting the growth of an aquatic photosynthetic organism within a growth medium in a photobioreactor may use a luminescent material targeting the aquatic photosynthetic organism in the photobioreactor. The luminescent material may be a substrate with a matrix of conductors coupled to the substrate, and light emitting diodes (“LEDs”) electrically coupled to the matrix of conductors. The aquatic photosynthetic organism in the photobioreactor is exposed to the light emitted by the LEDs.

### CROSS-REFERENCES TO RELATED APPLICATIONS

This application claims the benefit of U.S. Provisional Patent Application No. 61/550,912, filed Oct. 24, 2011 and U.S. Provisional Patent Application No. 61/578,107, filed Dec. 20, 2011, and incorporates the disclosure of each application by reference.

### BACKGROUND OF THE INVENTION

Terrestrial aquaculture of aquatic photosynthetic organisms may be optimized for a variety of purposes. Plants may be grown in a liquid growth medium for food, such as lettuce, radishes, and herbs. Photosynthetic bacteria may be grown for research purposes or for industrial applications, such as wastewater treatment. Algae may be grown to produce a variety of target products such as biofuels, nutritional supplements, food additives, and animal feed. The relatively high growth rate, high growth density, and high target product content of algae has inspired interest from the scientific community and spurred a new industry focused on developing the potential of this organism.

Optimizing the growth of such organisms in terrestrial systems such as open ponds or photobioreactors to produce in commercially viable quantities with the requisite quality of target product faces a number of challenges. Factors such as the expense of large quantities of water, the cost of land, the cost of providing nutrients, deficiencies in the various growth apparatus designs that may promote contamination and/or impede growth density, and challenges in harvesting and refining methods indicate that further developments in the growth of such organisms, especially algae, may enhance these organisms as environmentally and economically viable sources for target products such as biofuels.

### SUMMARY OF THE INVENTION

Methods and apparatus for growing an aquatic photosynthetic organism according to various aspects of the present invention may promote the growth of the aquatic photosynthetic organism within a growth medium. A luminescent material may provide light to the aquatic photosynthetic organism in a photobioreactor. For example, the luminescent material may comprise light emitting diodes, such as micro-scale light emitting diodes (“micro-LEDs”). The aquatic photosynthetic organism in the photobioreactor may be exposed to the light emitted by the micro-LEDs to promote the growth of the aquatic photosynthetic organism.

It is an object of the present invention to provide a multitude of lights to provide light to the photosynthetic organism to reduce shadows and refraction losses.

It is another object of the present invention to provide light to areas within the photobioreactor, which receive little light, such as the bottom of a pond, or are in darkness, such as in a pipe, or are in darkness because of night.

It is a further object of the present invention to provide a matrix of micro-LEDs having at least 10 micro-LEDs per square inch and preferably many more.

It is yet another object of the present invention to provide a flexible sheet of lights that can be submerged into an aqueous liquid for growing a photosynthetic organism.

It is yet a further object of the present invention to provide specific wavelengths of light or specific periods of light to the photosynthetic organism.

It is an additional object of the present invention to provide a photobioreactor with luminescent material incorporated into or on a wall of the photobioreactor.

It is yet another additional object of the present invention to provide a coating on a luminescent material to hold photosynthetic organism adjacent to the luminescent material.

### BRIEF DESCRIPTION OF THE DRAWING FIGURES

A more complete understanding of the present invention may be derived by referring to the detailed description when considered in connection with the following illustrative figures. Like reference numbers refer to similar elements and steps throughout the Figures.

Elements and steps in the Figures are illustrated for simplicity and clarity and have not necessarily been rendered according to any particular sequence or scale. For example, steps that may be performed concurrently or in different order are illustrated in the Figures to help to improve understanding of embodiments of the present invention.

The Figures described are for illustration purposes only and are not intended to limit the scope of the present disclosure in any way. Various aspects of the present invention may be more fully understood from the detailed description and the accompanying drawing figures, wherein:

FIG. 1 representatively illustrates the direction of flow of growth media in an exemplary photobioreactor;

FIG. 2 representatively illustrates a top view of a luminescent material;

FIG. 3 representatively illustrates a cross-sectional view of the luminescent material;

FIG. 4 representatively illustrates an image of an exemplary array of micro-LEDs;

FIG. 5 representatively illustrates the luminescent material applied to a v-trough type photobioreactor;

FIG. 6 representatively illustrates the luminescent material applied to a flat panel type photobioreactor;

FIG. 7 representatively illustrates the luminescent material applied to raceway pond type photobioreactor;

FIG. 8 representatively illustrates the luminescent material applied to a bubble column type photobioreactor;

FIG. 9 is a block diagram of an exemplary programmable control system;

FIG. 10 is an illustration of the rate of photosynthesis of an algae along a photosynthesis action spectrum;

FIG. 11 is an illustration of an absorption spectrum for chlorophyll and carotenoid photo-harvesting pigments;

FIG. 12 representatively illustrates an exemplary automated continuous flow photobioreactor regulated by the programmable control system;

FIG. 13 is a flow chart illustrating an exemplary method of operating the flexible luminescent material in the photobioreactor;

FIG. 14 is a flow chart illustrating a representative embodiment of a method of assembling the luminescent material;

FIG. 15 representatively illustrates the luminescent material applied to a substantially rigid substrate;

FIG. 16 representatively illustrates the substantially rigid substrate configured as a telescoping tube; and

FIG. 17 representatively illustrates the substantially rigid substrate configured as a sheet that may be adapted for insertion into a wall of the photobioreactor.

### DETAILED DESCRIPTION OF THE INVENTION

Methods and systems according to various aspects of the present invention may be described in terms of functional block components and various processing steps. Such functional blocks may be realized by any number of components configured to perform the specified functions. For example, the described methods and systems may employ various process steps, apparatus, systems, methods, etc. for implementing the functions and results. The described methods and apparatus may be practiced in conjunction with any number of systems and methods for promoting the growth of aquatic photosynthetic organisms, and the systems described are merely exemplary applications and embodiments for the invention. Various representative implementations of the present invention may be applied to any appropriate type of photobioreactor for an aquatic photosynthetic organism. Certain representative implementations may include, for example, applying a luminescent material to the photobioreactor to promote the growth of the aquatic photosynthetic organism.

The particular implementations shown and described are illustrative of the invention and its best mode and are not intended to otherwise limit the scope of the claimed invention in any way. For the sake of brevity, conventional manufacturing, connection, preparation, and other functional aspects of the system may not be described in detail. Furthermore, the connecting lines shown in the various figures are intended to represent exemplary functional relationships between the various elements and/or steps. Many alternative or additional functional relationships or physical connections may be present in a practical system.

Various aspects of the invention may provide methods, apparatus, and systems for promoting the growth of an aquatic photosynthetic organism within a growth medium in a photobioreactor. A detailed description of various embodiments for promoting the growth of an aquatic photosynthetic organism within a growth medium in a photobioreactor is provided as a specific enabling disclosure that may be generalized to any application of one or more aspects of the disclosed systems and methods in accordance with the various described embodiments and the claims. Various representative implementations of the present invention may be applied to any suitable photobioreactor for the cultivation of an aquatic photosynthetic organism, such as a plant, bacterium, and/or algae.

Certain representative implementations may include, for example, systems and methods for providing light to the aquatic photosynthetic organism using a luminescent material. Luminescent material is a material that provides light to the photosynthetic organism. It may be any device that utilizes electricity to provide light, either directly or indirectly. For example, fluorescent light, halogen light, incandescent light, light emitting diodes, polymer light emitting diodes, organic light emitting diodes, phosphor based LED, liquid crystal display (LCD), piezoelectric LED, etc. LEDs are traditionally made by group III and group V materials such as ZnSe, InGaN, GaN, GaP, AlGaInP, AlGaP, GaAsP, AlGaAs, etc. Modern nanocrystal or quantum dot LEDs may also be used. Other luminescent materials may be made with ZnS doped with copper, silver or manganese. For the purposes of this specification, the term LED will be used generically to all of the different types of LEDs and related luminescent semiconductor devices and such LEDs made small enough constitute micro-LEDs. White OLEDs emit white light that is brighter, more uniform and more energy efficient than that emitted by fluorescent lights. OLEDs can also be made is large sheets. White OLEDs also have the true-color qualities of incandescent lighting.

Examples of indirectly produced light include the same light sources optically connected to fiber optics or a light pipe. Ambient and solar light provided by a light pipe to the luminescent material is also an embodiment of the present invention. Other light sources such as a xenon flash lamp, laser, etc may be used to provide light carried by optical fibers. The optical fibers may terminate at the luminescent material or they may be shaped to have a flat or irregular portion that allows light to escape the optical fiber, or the non-terminal portion of an optical fiber may be made of a different composition, which “leaks” light by not providing complete internal reflection and/or refraction of light. The optical fibers may terminate at a point in the luminescent material, travel partially or completely through the material by a straight, serpentine or woven manner. For example, optical fiber fabric, a product known per se, may be used as a luminescent material. The luminescent material may be a sheet of partial internal reflecting and or refracting light transferring material, which emits light provided via an optical fiber or a light source. The luminescent material may be a monolithic solid or it may be porous, allowing fluids, such as culture medium, to pass through it.

The luminescent material may form a mat that has a porous component with increase surface area. In this arrangement, attachment of photosynthetic organism, such as algae, to form a biofilm is desired. Translucent or transparent plastic or glass fibers are attached to the luminescent material forming a thick wooly coating on the luminescent material, which is constantly exposed to the culture liquid. This mat may be partially or wholly submerged in culture medium or even completely exposed provided that it is hydrated. The photosynthetic organism is harvested by scraping or washing the mat. Open air mats may be used for growing algae such as those in lichens and the like.

In an exemplary embodiment, the luminescent material may be adapted for use with a photobioreactor to target the aquatic photosynthetic organism within the growth medium for exposure to the light emitted by the luminescent material. In one embodiment, the luminescent material may comprise light-emitting diodes (LEDs) positioned within or near the photobioreactor to target the aquatic photosynthetic organism with light. Certain representative implementations may also include other components in addition to the luminescent material, such as a programmable control system for regulating the function of the LEDs, and/or environmental sensors, such as photocell sensors for providing data about light intensity, such as from the LEDs or sunlight.

In one embodiment, the luminescent material may provide a customized light wavelength and/or intensity to photo-harvesting pigments of the aquatic photosynthetic organism, for example to promote growth. In one exemplary embodiment, the luminescent material may supplement ambient light, such that the luminescent material may optimize the growth conditions of the aquatic photosynthetic organism by providing light in locations of the growth apparatus that ambient light, whether it is sunlight or from an artificial light source, cannot effectively reach. For example, the outer surfaces of the growth apparatus may be shaded from ambient light, such as along a bottom surface or an angled surface. Further, the aquatic photosynthetic organism being cultivated inside the photobioreactor may transiently occupy space having diminished levels of light.

In some embodiments, supplementing ambient light with light emitted from the luminescent material may reduce an operation cost of the photobioreactor, such as by reducing or eliminating the need for alternate artificial light and/or increasing the density of the aquatic photosynthetic organism such that fewer cultures or smaller cultures may be cultivated. In addition, supplementing ambient light with the light emitted from the luminescent material may increase or otherwise improve growth and/or photo-harvesting pigment concentration in the aquatic photosynthetic organism. In other embodiments, the luminescent material may comprise the primary light source, and further may be powered by any appropriate energy source, such as photo-electricity, hydropower, wind power, coal power, nuclear power, and/or petroleum power.

Light may be provided continuously or intermittently based on photobioreactor design, type of photosynthetic organism used and product produced. Most photosynthetic organisms have a photosynthetic receptor and enzyme system that becomes saturated by continuous light and must wait several to many milliseconds to desaturate and reset before adsorbing more light. Artificially produced light may be provided more efficiently by exposing the photosynthetic organism to a flashing light as providing more light to a saturated photosynthetic system does not enhance photosynthesis. Over exposure to light causes stress on the photosynthetic organism, which may reduce product production, and greater stress leads to bleaching and death.

While sunlight, ambient light and certain types of artificial light are continuous; certain types of luminescent material can provide flashing light in the millisecond rate to optimally enhance photosynthesis without wasting energy. LEDs and other semiconductor-based lights can flash at very high rates and be fine tuned to the particular photosynthetic organism, its stage in life and the desired target product produced. Typically, an OLED can flash faster than a conventional. Flashing light may be effectively provided from a constant light source by a number of methods. For example, continuous light may be shined on a spinning mirror or other optical gate, which provides light intermittently. Alternatively, continuous light may be shined in a pattern within a photobioreactor or continuously moved so that any single photosynthetic organism receives the shined light intermittently. Also, the photosynthetic organism may move with respect to a stationary light, such as being suspended in a liquid, which flows across a light. For example, by using a band of luminescent material or bands of light sources within a luminescent material in a tube or raceway, the photosynthetic organism passes by each continually shining light every few to many milliseconds to provide a flashing effect. The light exposure or non-exposure times during flashing may be constant, variable or random as desired and in accordance with the photobioreactor design. Furthermore, fiber optics may be incorporated such that the flashing may be caused by a device external to the photobioreactor, such as by a flash lamp or laser. Fiber optics may also be used to provide an indirect flashing effect in the same manner as described above.

The luminescent material may include different colored lights (e.g. different LEDs) or the same light providing different wavelengths of light in a flashing manner. One particular wavelength or group of wavelengths may be flashed at a different rate than another. Should the photosynthetic organism have different pigments that saturate and desaturate at different rates, the flashing lights may be so adjusted to provide optimal light energy with minimal energy usage.

Optical filters may be applied on top of individual micro-LEDs, arrays of micro-LEDs, the surface of optical fibers at either end or inside or outside the photobioreactor, or the entire luminescent material to avoid treatment with undesirable wavelengths which may be harmful to the photosynthetic organism, alter its metabolism or provide unwanted excess heat to the photosynthetic organism. An optical filter may also be used to control the wavelengths of light provided by ambient or sunlight.

In various embodiments, the aquaculture of the aquatic photosynthetic organism may be performed in a photobioreactor. The photobioreactor may comprise any suitable device or apparatus adapted to support a biologically active environment, such as a marine and/or terrestrial apparatus for containing the aquatic photosynthetic organism with a growth medium to promote its growth. In one embodiment, the photobioreactor may provide light, whether the light is ambient light and/or artificial light, to the aquatic photosynthetic organism in the photobioreactor. Configurations of the photobioreactor may include, but are not limited to, an open pond (natural or man-made), a raceway pond, a tubular photobioreactor, a closed tank photobioreactor (also referred to as barrels), a bag system, a v-trough, a bubble column, and/or a flat-panel photobioreactor.

The luminescent material may be shaped to conform to any part of a photobioreactor such as a surface of an open pond (natural or man-made), a raceway pond, a tubular photobioreactor, a closed tank photobioreactor (also referred to as barrels), a bag system, a v-trough, a bubble column, and/or a flat-panel photobioreactor. The luminescent material may form a tube through which photosynthetic organism within the culture medium may travel such as to supplement illumination or to provide one or more wavelengths for a particular purpose. The luminescent material may be manufactured as the photobioreactor, often by increasing the thickness. The luminescent material may be manufactured as a rigid liner or a rigid wall of a photobioreactor. “The luminescent material may be a divider or be attached to or be part of a divider in the photobioreactor. Different sides of the divider may have the same, different phases in the growth cycle or different photosynthetic organisms or to increase the amount of available light at the point of insertion.” The divider can be used to create sections within the photobioreactor or to increase the amount of light available to the photosynthetic microorganism in any part of the photobioreactor.

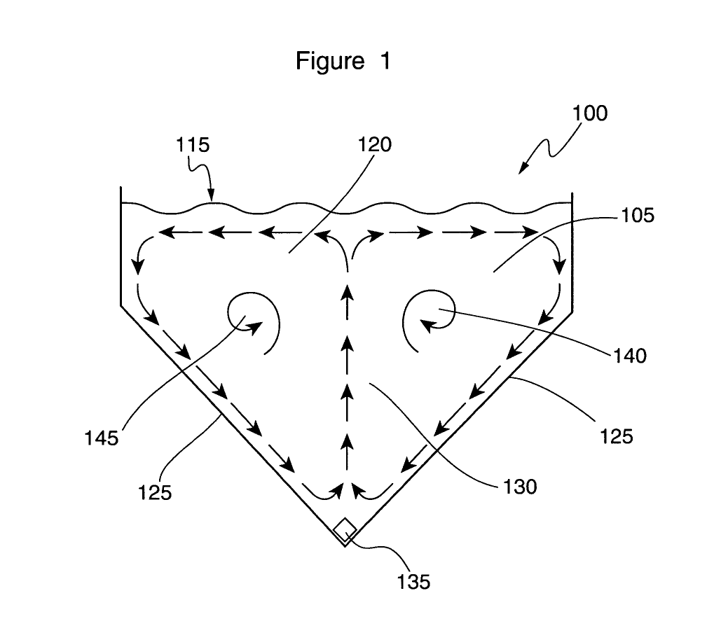

For example, referring to FIG. 1, an exemplary v-trough shaped photobioreactor 100 may contain an aquatic photosynthetic organism, such as algae, growing in a growth medium 105. The algae may circulate, such as in a clockwise pattern 140 and/or a counterclockwise pattern 145, that may be produced by an aerator 135 or other circulator. In one embodiment, the top surface 115 of the photobioreactor 100 and/or the growth medium 105 may receive ambient light, such as sunlight, during at least a portion of a day. In some embodiments, one or more parts of the photobioreactor 100 may not receive direct sunlight for at least part of the day. For example, algae near the angled surfaces 125 and/or a vertical area 130 may not be directly exposed to the sunlight for at least part of the day. In some embodiments, only the algae in a horizontal area 120 near the surface may be directly exposed to the sunlight. Accordingly, due to inconsistent sunlight exposure, the algae circulating near the angled surfaces 125 and the vertical area 130 may not grow at an optimal photosynthetic rate until they reach the horizontal area 120, which may only be a fraction of the area the algae occupies as it circulates through the photobioreactor 100.

Further, in one embodiment, the growth rate and/or density of the aquatic photosynthetic organism may be limited due to absorption of ambient light by the growth medium 105, which may diminish the penetration of the ambient light through the growth medium 105. The aquatic photosynthetic organism may also self-shade as the density of the culture increases (i.e., one organism may shade another nearby organism from the ambient light), further diminishing the amount of ambient light reaching all of the aquatic photosynthetic organisms in the photobioreactor.

Various aspects of the present invention may promote the growth of any suitable aquatic photosynthetic organism. The aquatic photosynthetic organism may comprise any appropriate organism that may grow and/or propagate in a liquid growth medium. In one embodiment, the aquatic photosynthetic organism may comprise a plant grown by hydroponic or aquaponic methods in a growth medium comprising a nutrient solution in water without the presence of soil. For example, at least a portion of the plant, such as the roots, may be submerged in the growth medium for absorption of nutrients. In another embodiment, the aquatic photosynthetic organism may comprise a photosynthetic bacterium. For example, the photosynthetic bacterium may comprise bacteria from any of the five bacterial phyla of Chlorobi, Chloroflexi (filamentous anoxygenic phototrophs), Firmicutes (heliobacteria), Proteobacteria (purple sulfur and purple nonsulfur bacteria), and Cyanobacteria (sometime referred to as “blue-green algae”). In an exemplary embodiment of the present invention, the photosynthetic bacteria may comprise purple non-sulfur bacteria used in the aquaculture of probiotics, as well as for the degradation of organic wastes.

In one embodiment according to various aspects of the present invention, the aquatic photosynthetic organism may comprise a species and/or mixture of species of algae. The algae may comprise any one or more of the thousands of algal species now know or hereinafter discovered. For example, in various embodiments, the algal species may comprise microalgae, macroalgae, and/or marine microalgae and macroalgae. The aquaculture of the aquatic photosynthetic organism may be performed in the photobioreactor 100 and the growth medium 105. The growth medium 105 may comprise any suitable medium for facilitating growth of the photosynthetic organism, such as a gas, liquid, gel, solid, or a combination thereof, like a liquid including a particulate suspension or a solid saturated with fluid. In one embodiment, the growth medium 105 may comprise water and any nutrients and/or dissolved gasses that the aquatic photosynthetic organism may use for growth and/or production of a target product(s). For example, the growth medium 105 may comprise a carbon source, such as dissolved carbon dioxide gas (CO₂(g)) or bicarbonate. In another embodiment, the growth medium 105 may also comprise nutrients such as phosphorus, nitrogen, iron, and/or sulfur. In an exemplary embodiment, the nutrients may be provided by an agricultural fertilizer and/or an organic waste such as dead leaves, grass clippings, and/or animal waste. In one embodiment, the nutrients may be provided by a wastewater treatment plant that may utilize anaerobic bacteria to digest and breakdown the waste.

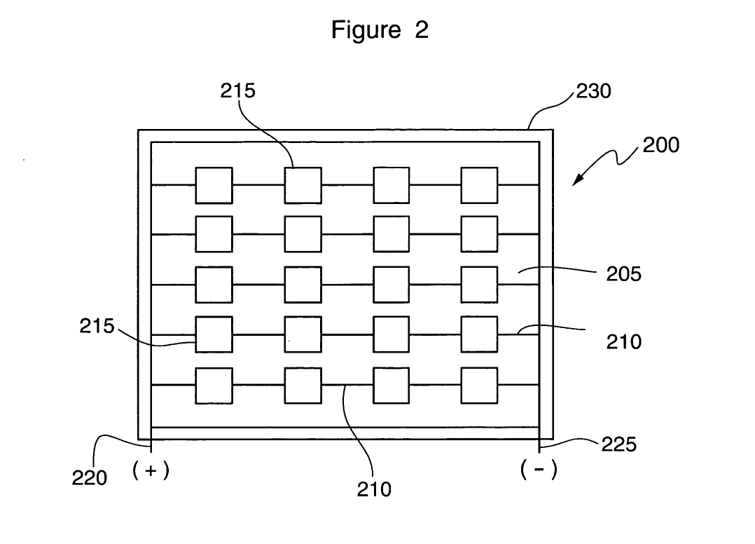

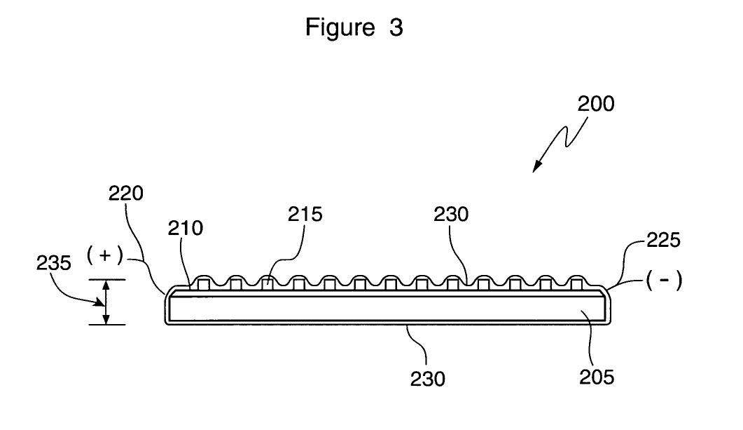

The luminescent material provides light to facilitate growth of the photosynthetic organism. The luminescent material may comprise any appropriate mechanism for providing light to the photosynthetic organism in the photobioreactor 100, such as incandescent lights, LEDs, reflectors, refractors, and/or other appropriate mechanisms for generating and/or otherwise providing light. For example, referring to FIGS. 2 and 3, an exemplary luminescent material 200 may comprise a light source 215, such as a light-emitting diode (LED), on a substrate 205. The luminescent material 200 may further comprise other elements, such as a matrix of conductors 210 and/or a protectant 230. The light source 215 may receive power, for example from a power source electrically connected to the matrix of conductors 210. Any appropriate elements may be connected to the luminescent material 200 and/or powered through the matrix of conductors 210, such as additional light sources, power sources, cooling systems, sensors, transmitters, control systems, and/or other components. In various embodiments, the luminescent material 200 may comprise flexible materials to form a flexible luminescent material.

The substrate 205 supports one or elements of the luminescent material, such as the light sources 215 and the conductors 210. In the present embodiment, the substrate 205 may comprise any appropriate material that may be adhered or otherwise attached to the matrix of conductors 210, the light sources 215, and/or a surface of the photobioreactor 100. In one embodiment, the substrate 205 may comprise a substantially electrically nonconductive material. For example, the substrate 205 may comprise a polyester film, such as Mylar® brand polyester films.

In some embodiments, the substrate 205 may comprise a flexible material, such as one or more of an acetate film, vinyl sheet, polymer substrate, plastic, and/or composite. Any material that is transparent or translucent to emitted light and permits the luminescent material sufficient flexibility to be bent to form a cylinder may be used. The substrate 205 comprising a flexible material may have any suitable thickness or other dimensions. In one embodiment, the luminescent material 200 may be regarded as a thin film having a predetermined thickness 235. The thin film may have the appearance and/or characteristics of a thin skin, a membrane, and/or a thick skin. The predetermined thickness 235 may be uniform or may vary across the luminescent material 200. In one embodiment, the predetermined thickness 235 may be approximately 0.0001 millimeter (mm) to 2.0 mm. In another embodiment, the predetermined thickness 235 may be approximately 0.1 mm to 2.0 mm. In another embodiment, the predetermined thickness 235 may be approximately 0.01 mm to 2.0 mm. In yet another embodiment, the predetermined thickness 235 may be approximately 0.001 mm to 2.0 mm. Thickness can be determined by the desired flexibility required of the product or by the thickness of the elements in the product.

In various embodiments, the substrate 205 may comprise a substantially rigid substrate. For example, the substantially rigid substrate may comprise glass, ceramic, fiberglass, plastic, polymerized vinyl chloride, crystalline material and/or a combination thereof.

The placement of plural luminescent material 200 inside the photobioreactor 100 should be at intervals at or beyond the normal penetration of emitted light. For example, if light emitted by the luminescent material effectively penetrates 20 centimeters, multiple rigid or flexible luminescent material may be submerged in the photobioreactor culture medium at roughly parallel zones approximately 400 centimeters apart. Since different photosynthetic organisms, different culture mediums and different stages in organism growth all affect the amount of light penetration, it may be advantageous to have the luminescent material 200 located at adjustable positions inside the photobioreactor 100. Reflectors or a reflective surface inside the photobioreactors also determine location of the luminescent material 200.

The substrate 205 may exhibit other mechanical and/or physical properties such as shapeability, stretchability, mechanical ruggedness, anti-corrosive properties, anti-biofouling properties, optical transparency, and/or combinations thereof. For example, the substrate 205 may comprise an anti-biofouling agent that may deter or kill microorganisms that may attach to the luminescent material 200, which might otherwise disrupt the optical properties of the luminescent material 200 and/or cause damage to its components. For example, the anti-biofouling agent may comprise a biocide such as the salts, chelates and compound derivatives of copper, tin, silver, titanium, mercury and arsenic as well as organic quaternary ammonium based and isothiazolinone based compounds. The anti-biofouling agent may be absorbed by the substrate 205 and/or may be applied to the surface of the substrate 205, such as via paint or other coating. A movable divider in the photobioreactor with the luminescent material on it may be present in the photobioreactor so that it can accommodate different amounts of light penetration as the culture changes while maintaining optimal light exposure.

The matrix of conductors 210 supplies electricity or light to the light sources 215. The matrix of conductors 210 may comprise any suitable optical, electronic, or related components such as fiber optics, electrical interconnects, electrodes, insulators, resistors, electronic elements, and electro-optical elements. For example, the matrix of conductors 210 may comprise conductive wires, nanowires, and/or nanotubes to supply electricity to the light sources 215 or optical fibers to transmit light to the light sources 215. In one embodiment, the matrix of conductors 210 may be electrically coupled to at least one of the power source and the programmable control system, providing power to and/or control of the light source 215.

The matrix of conductors 210 may be coupled to the substrate 205 by any suitable mechanism and/or method. For example, a variety of techniques in the field of microelectronics may be used to couple the matrix of conductors 210 to the substrate 205, including but not limited to ink jet printing adjusted to apply a mixture of wires with an adhesive substance onto the substrate 205, conventional silkscreen printing techniques, optical photolithography, deposition techniques (e.g., chemical vapor deposition, physical vapor deposition, atomic layer deposition, sputtering deposition etc.), and/or soft lithography (used to create a conductive pattern). The electrical connections to the LED or other light source need not be a thin wire as exemplified. Rather it may constitute one or more layers. For example thin conductive layers for the cathode and anode may sandwich the semiconductor layer. Transparent cathode and anode layers are known in the art per se. Transparent substrates and transparent OLEDs and other luminescent devices are known in the art per se and may be used. This permits the light source to shine in two directions, which is preferred when the luminescent material is submerged in the photobioreactor.

The light source 215 provides light to facilitate growth of the organism in the growth medium 105. The light source 215 may comprise any suitable light-generating system adapted to generate and/or transmit light. For example, the light sources 215 may comprise solid-state lights receiving power from the matrix of conductors 210. The light sources 215 may also comprise or be connected to components for operating the light sources 215. For example, the light source 215 may be connected to an input voltage conversion unit that accepts any anticipated AC or DC voltage and converts the input into an appropriate supply signal, such as a DC voltage that powers the solid state light. The light source 215 may also comprise a current source and a solid-state high power light and/or any other appropriate elements for generating or transmitting light.

The light source 215 may be adjusted or selected according to any appropriate criteria. For example, the light source 215 may exhibit a high electrical efficiency, high thermal conductivity, reliability, long operating lifetime, and/or an optimal size for the desired application. In various embodiments, the light source comprises multiple LEDs, such as micro-LEDs, generating light in response to an electrical signal. The LEDs may be obtained from conventional commercial manufacturers such as, but not limited to, CREE, Philips Electronics, and Epistar Corporation. Micro-LEDs may require significantly less power than conventional LEDs. For example, micro-LEDs may require a few billionths of an ampere to operate. The low power requirement may reduce energy costs and operational costs of powering the luminescent material 200. Micro-LEDs may also produce less heat than conventional LEDs.

The distinction between micro-LEDs and LEDs is one of size and arrangement. Other features attributed to LEDs also apply to micro-LEDs. Micro-LEDs are generally closely and evenly spaced. This permits fewer areas in the shadows and fewer areas exposed to too much light in photobioreactors using them. In the present invention, the size, density and number of micro-LEDs depend on the photosynthetic organism and its culture density. In the preferred example, Nannochloropsis sp. is used and is approximately 2 microns in diameter. A luminescent material with a micro-LED sized similar to the algae cell would be advantageous.

For the purpose of this application, some of the uses may use either and with others, the distinctions between the two terms become blurred. For example an array of large LEDs used with diffuser lenses may function similar to a micro-LED. However, micro-LEDs are not to be confused with conventional LED light bars used for indoor illumination.

The LEDs may comprise any suitable materials or configuration to provide the light. In various embodiments, the LEDs may comprise gallium-based crystals such as gallium nitride, indium gallium nitride, and/or gallium aluminum phosphide. The LEDs may further comprise an additional material, such as phosphorus to produce white light. For example, a phosphor material may convert monochromic light from a blue or UV LED to broad-spectrum white light.

The LEDs may comprise, however, any suitable LED system. The LEDs may be flat, a cluster, and/or a bulb. The LEDs may emit white light, colored light, or combinations of different wavelengths, frequencies, intensities, and/or polarizations. For example, the LEDs may provide a light intensity output of approximately 1-6000 μmol of photons per square meter per second. In one embodiment, the LEDs may be configured to provide a light intensity output of approximately 1-4000 μmol of photons per square meter per second. In another embodiment, the intensity of the light emitted from the LEDs may be adjusted to be less than a maximum irradiance tolerance of the aquatic photosynthetic organism.

The light source 215 may be electrically coupled to the matrix of conductors 210. For example, the LEDs may be printed on the substrate 205 and the matrix of conductors 210 using a conventional ink jet printing system. In one embodiment, the micro-LEDs may be printed onto the substrate 205 with a silver epoxy material to facilitate the electrical connection.

Each LED may comprise at least one positive electrode 220 and at least one negative electrode 225. The positive electrode 220 and the negative electrode 225 may be coupled to the matrix of conductors 210. In one embodiment, the LEDs may be electrically coupled through the matrix of conductors 210 to at least one of the power source and the programmable control system, providing power to and/or control of the LEDs.

The LEDs may be arranged in a selected pattern in the photobioreactor to generate various combinations of light exposure. For example, the LEDs may be positioned, flashed, or otherwise varied in a strategic pattern in the photobioreactor to generate various combinations of light exposure in different areas of the photobioreactor. In another embodiment, the LEDs may be arranged in the photobioreactor in a strategic pattern in combination with flashing or otherwise varying light to generate various combinations of light exposure.

In one embodiment, the light source 215 may comprise different sets of micro-LEDs that emit light at the same or different wavelengths. For example, a first set of micro-LEDs may emit light in a blue wavelength, a second set of micro-LEDs may emit light in a red wavelength, and/or a third set of micro-LEDs may emit light in a far red wavelength. In various embodiments, any set of micro-LEDs may be configured to emit light of any appropriate wavelength, such as ultraviolet and infrared. For example The LEDs may comprise any suitable LED or combination of LEDs, such as a blue-green-red-far red LED system, micro-LEDs, and/or a phosphor-converted LED, which may produce the desired appropriate wavelength(s) even when the underlying LED, without a phosphor, does not.

A laser delivered to the photobioreactor by a bundle of optical fibers deliver only one wavelength of light and may be preferred over the narrow band of light from a LED.

In some instances, it may even be beneficial to emit a wavelength outside the normal or needed ranges as these other wavelengths may have other beneficial or metabolism altering effects on the photosynthetic organism or other organisms in culture. For example, ultraviolet-C may be used to kill bacteria and other microbial contaminants in the culture medium. The same or other wavelengths catalyze various chemical reactions and degrade various compounds (with or without the presence of a chemical catalyst) to effectively remove them. For example, contaminated or waste water and photosynthetic organism waste and byproducts of the culture may be inactivated or degraded using UV or visible light alone or in conjunction with a photocatalyst in a process known per se outside the field of culturing photosynthetic organisms. These are also considered contaminants because they are unwanted, even if produced inside the photobioreactor. Selective wavelengths and amounts may adequately reduce the number or growth rate of contaminants without causing excessive harm to the photosynthetic organism or the desired target product(s). It may even be beneficial to continually mutate the photosynthetic organism using UV (in small amounts), to provide a continual artificial evolution of more rapidly growing and better photobioreactor adapted photosynthetic organism. When combined with product measurements and selection of higher producers, artificial evolution of higher product producing photosynthetic organisms may result, particularly with respect to that, and other, photobioreactor systems.

The LEDs may be distributed in a horizontal plane onto the matrix of conductors 210 in an array of individual LEDs at any appropriate density. The array of LEDs on the matrix of conductors 210 may be arranged in at least one of a regularly spaced array, a disordered array, or a combination thereof. The LEDs may comprise a group, array, and/or sheet of LEDs that may be diced or otherwise separated into individual LEDs for coupling to the matrix of conductors 210.

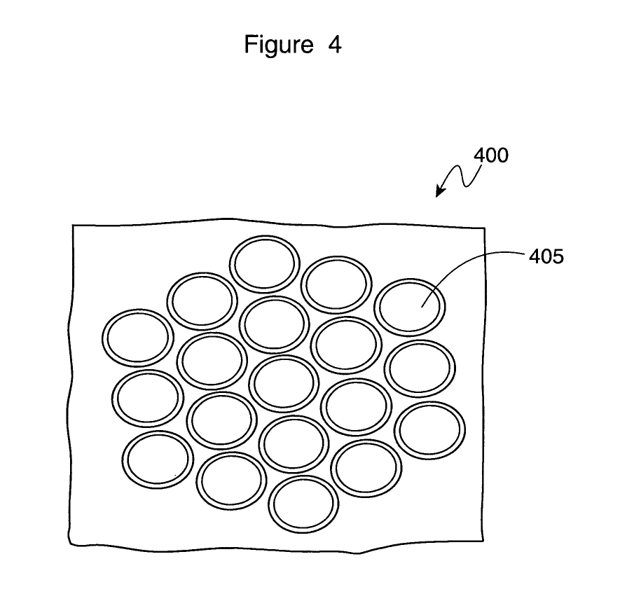

In one embodiment, the LEDs may comprise micro-LEDs. Micro-LEDs may be significantly smaller than conventional LEDs. In an exemplary embodiment, each micro-LED may be approximately 15 μm in diameter. In another embodiment, the LEDs may comprise an array of micro-LEDs. For example, referring to FIG. 4, the micro-LEDs may be provided as a 19-pixel micro-array 400 wherein each micro-LED 405 may be approximately 14 μm in diameter. The micro-LEDs may be diced or otherwise separated into individual micro-LEDs for subsequent coupling to the matrix of conductors 210. In one embodiment, the micro-LEDs may be arranged in strategic clusters and/or patterns to maximize production of the target product(s).

In another embodiment, the micro-LEDs may be arranged in strategic clusters and/or patterns in combination with intermittent light to maximize production of the target product(s).

In some embodiments, the predetermined density of LEDs may be arranged in densities of approximately 1 to 1 million micro-LEDs per square inch of the matrix of conductors 210. In one embodiment, the predetermined density of LEDs may provide uniformity of light intensity. For example, tightly packed micro-LEDs may emit a wall of substantially uniform light at a desired intensity that may promote the growth of the aquatic photosynthetic organism passing by the micro-LEDs as the aquatic photosynthetic organism circulates through the photobioreactor. In contrast to conventional LED light systems, such as decorative LED lighting, the wall of substantially uniform light may lack dark areas between lighted areas to maintain the substantially uniform wall of light and maintain a substantially constant influx of photons at the desired intensity to the aquatic photosynthetic organism for photosynthesis.

In one embodiment, the micro-LEDs may be arranged in densities of 1-10 per square inch of the matrix of conductors 210. In another embodiment, the micro-LEDs may be arranged in densities of 10-100 per square inch of the matrix of conductors 210. In yet another embodiment, the micro-LEDs may be arranged in densities of 100-1000 per square inch of the matrix of conductors 210. In yet another embodiment, the micro-LEDs may be arranged in densities of approximately 1,000-10,000 per square inch of the matrix of conductors 210.

Additionally, the predetermined density of the micro-LEDs on the matrix of conductors 210 may be in a density of greater than 25 micro-LEDs per square inch of the matrix of conductors 210, such as approximately 25 to 100 micro-LEDs per square inch of the matrix of conductors 210. In another embodiment, the predetermined density of the micro-LEDs on the matrix of conductors 210 may be in a density of at least one of less than approximately 10,000 micro-LEDs per square inch, less than approximately 100,000 micro-LEDs per square inch, less than approximately 1,000,000 micro-LEDs per square inch, and/or greater than approximately 1,000,000 micro-LEDs per square inch. Large numbers of tiny light sources in high density are readily achieved and may be made by a number of known techniques known in the art, particularly those in the semiconductor field such as photolithography. For an example of using tiling, see U.S. Pat. Nos. 5,837,832 and 5,744,305.

In various embodiments, the protectant 230 may comprise any material and/or composition that may inhibit liquid, dust, and/or moisture from contacting the light source 215, the matrix of conductors 210, and/or the substrate 205. In some embodiments, the protectant 230 may inhibit damage to the luminescent material 200 from corrosion, electrical damage, and/or electrostatic discharge. In addition, the protectant 230 may transmit, diffuse, focus, polarize, and/or filter the light emitted by the light source 215.

In various embodiments, the protectant 230 may comprise a waterproof material covering a top surface or all surfaces (e.g., encapsulation) of the luminescent material 200. The protectant 230 may be configured such that the positive electrode 220 and/or the negative electrode 225 may reach the power source. For example, the protectant 230 may be located on the top surface of the substrate 205 where the conductive matrix 210 and the light source 215 may be located. In one embodiment, the protectant 230 may comprise a thin film of transparent or translucent material that may be printed, coated, or otherwise transferred onto the substrate 205 along with the light source 215. In another embodiment, the protectant 230 may encapsulate the luminescent material 200. For example, the protectant 230 may comprise a liquid polymer in which the luminescent material 200 may be dipped and that subsequently cures or dries. In another embodiment, the protectant 230 may comprise a material that may be laminated onto the luminescent material 200. In various exemplary lamination processes, the protectant 230 may be rolled or pressed onto the luminescent material 200, such as by a cold lamination process.

In an exemplary embodiment according to various aspects of the present invention, the protectant 230 may comprise any suitable material that may at least partially seal the luminescent material 200 and may exhibit desirable optical characteristics, such as a high transmittance of the light emitted from the light source 215. For example, the protectant 230 may comprise adhesives, plastic, silicon sealants, an epoxy resin, a polyurethane material, liquid plastic molds, high density polyethylene welds, polyethylene laminates, polycarbonate and the like.

In an alternative embodiment, the protectant is not tightly bound to the luminescent material but rather is a sleeve or container for the luminescent material. The luminescent material is placed inside the protectant and is preferably sealed closed. If the luminescent material with protectant is not completely submerged in culture medium, the protectant may not be sealed. The protectant preferably contains an anti-biofouling agent as mentioned below. The protectant may be permanent such as a hollow baffle or tube in the culture medium or it may be separated from the culture medium along with the luminescent material inside. This protectant may even be disposable, particularly if it does not contain an anti-biofouling agent described below.

In an exemplary embodiment, the protectant 230 may exhibit other useful mechanical and/or physical properties such as shapeability, stretchability, mechanical ruggedness, anti-corrosive properties such as from exposure to salt and/or salt water, anti-biofouling properties and/or combinations thereof. For example, the protectant 230 may comprise an anti-biofouling agent that may deter or kill microorganisms that may attach to the protectant 230, thereby obscuring its optical properties. The anti-biofouling agent may comprise a biocide such as the salts, chelates and compound derivatives of copper, tin, chrome, silver, mercury and arsenic as well as organic quaternary ammonium based and isothiazolinone based compounds. The anti-biofouling agent may be physically absorbed by the protectant 230 and/or may be embedded in or on the surface of the protectant 230.

In addition to an anti-biofouling agent or instead of an anti-biofouling agent, the protectant 230 may be composed of or contain an outer layer of material that is difficult to foul or is easily cleaned or permits use of harsher cleaning techniques. For example, a hydrophilic surface or one that displays a charge may take longer to foul while being acceptable to the photosynthetic organism. Also, smooth glass surfaces are generally easier to clean than plastic and may be cleaned by stronger acid (except HF), alkali, and solvents and at higher temperatures than plastics. Alternatively, a protectant with multiple layers, a pealable, dissolvable, easily abraded or thin layer scrapped off or decayed off (e.g. etching, heat or chemical degradation) may be used.

The luminescent material 200 may comprise additional active and/or passive elements including, but not limited to, sensors, optical components, dielectric structures, conductive structures, adhesive layers or structures, connecting structures, encapsulating structures, lens structures, light diffusing structures, reflectors, waveguides, optical coatings, light polarizing structures, wavelength filters, electro-optical elements, and/or thin film structures and arrays of these structures. In some embodiments, the active and/or passive device elements may be provided on the substrate 205.

The luminescent material 200 may be electrically coupled to any suitable power source for providing power to the light source 215 and/or other components such as other light sources, cooling systems, sensors, transmitters, and/or control systems. In various embodiments, the power source may supply power generated from conventional AC electrical service, solar cells, wind generators, and/or hydroelectric systems. The power source may comprise any suitable related elements, such as transformers, connectors, filters, conditioners, converters, resistors, and the like. In one embodiment, the power source may comprise one or more step down transformers for converting conventional 120V or 277V supply voltages to 24V for use by the light source 215 and/or the other components. The power source may also comprise any other appropriate elements, such as a backup battery and/or a portable electrical generator. For example, the backup battery and/or the portable electrical generator may provide emergency power to the luminescent material 200 when the primary power source is unavailable.

In some embodiments, the luminescent material 200 may be cooled such that the heat generated by the light source 215 may be dissipated, such as to prevent heat damage to the luminescent material 200 and/or to the aquatic photosynthetic organism. In one embodiment, the luminescent material 200 is submerged into the growth medium within a photobioreactor and the growth medium itself may adequately cool the luminescent material 200. In another embodiment, the luminescent material 200 may be inserted into the sleeve in the wall or attached to the wall of the photobioreactor, which may be air conditioned and/or air vented (not shown). A cooling system may also cool the luminescent material and/or dissipate heat away from the growth medium. For example, the cooling system may apply a coolant or airflow to the luminescent material.

The luminescent material 200 may be used with any suitable natural or man-made photobioreactor for the cultivation of the aquatic photosynthetic organism. The luminescent material 200 may be used with the photobioreactor in any suitable manner that may promote the growth of the aquatic photosynthetic organism grown in the photobioreactor. For example, the luminescent material 200 may be located on the inside or the outside (if the photobioreactor has a transparent or translucent portion) of the photobioreactor.

In various embodiments, the luminescent material 200 may be applied to the photobioreactor using adhesives, suction cups, hooks, and/or other connectors or fasteners to attach the luminescent material 200 to the photobioreactor. In one embodiment, the aquatic photosynthetic organism may be located at a pre-selected distance from the luminescent material 200, such as where the luminescent material 200 is located on the outside of a transparent photobioreactor or in a sleeve located in a transparent wall of the photobioreactor. In this case, the aquatic photosynthetic organism may be located at a distance of a few millimeters to several feet from the luminescent material 200. When activated, the light source 215 in the luminescent material 200 may distribute light onto the aquatic photosynthetic organism. The distributed light may be uniform or variable over time and/or distance, for example according to the placement of the light sources 215 and/or control of the light sources 215 by the control system.

In some embodiments, the aquatic photosynthetic organism may be flowing directly over the luminescent material 200, such as where the luminescent material 200 is submerged in the growth medium within a photobioreactor. For example, the luminescent material 200 may be submerged in the growth medium and may be coupled to a liner for or a component of the photobioreactor such as a wall, a mechanical mixer, a paddle wheel, a baffle, an aeration mechanism, and/or a weir. This is particularly useful when the vessel shell of the photobioreactor is opaque to outside light. The luminescent material may be clustered in areas with poor exposure to light or it may be diffused as a supplement to or replacement for ambient, sunlight or exterior provided light. In another embodiment, the luminescent material 200 may lie on the bottom of the photobioreactor and/or may be coupled to an object that lies on the bottom or floats in the growth medium in the photobioreactor. For example, a length of polyvinyl chloride (PVC) pipe may be covered with the luminescent material 200 and/or the luminescent material 200 may be rolled into a transparent tube or other configuration and placed inside the photobioreactor, such as floating on top of the growth medium, secured in the middle of the water column, or lying on the bottom of the photobioreactor. In another embodiment, the luminescent material 200 may be submerged in the growth medium to form structures that direct the flow of the growth medium. In yet another embodiment, the luminescent material 200 may be secured in the water column of the photobioreactor and may be configured to have light emitted from any side of the luminescent material 200, such as where the luminescent material 200 is folded. When an exterior portion of the photobioreactor allows light to enter, the luminescent material is preferably located away from an open, transparent or translucent portion of the photobioreactor when light is entering from the outside. However, when light is not entering, such as at night, the luminescent material may be located in, on or adjacent to the open, transparent or translucent portion of the photobioreactor.

In photobioreactor systems with liquid being pumped to various locations, the pipes typically are opaque or travel through dark areas such as underground. The pipes may be wrapped or lined with the luminescent material of the present invention. Liquid inside the pipes may be mixed to permit effective exposure to emitted light. For example, a fixed or movable spiral or flexible film may be located inside the pipes. The spiral or flexible film structure may be the luminescent material of the present invention or it may be attached to it. The same could be used in any dark area within the photobioreactor system.

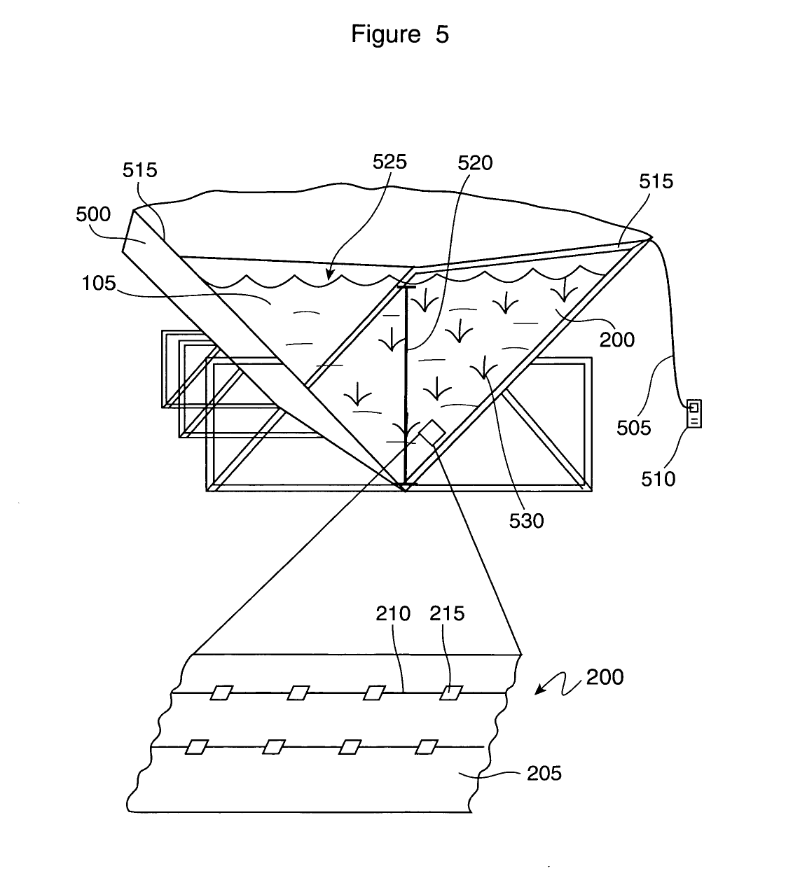

Referring to FIG. 5, in an exemplary embodiment of the present invention, the luminescent material 200 may be used with a v-trough type photobioreactor 500 for promoting the growth of algae in the growth medium 205. The luminescent material 200 may be attached to one or more inner surfaces 515 of the v-trough type photobioreactor 500, or ever be a liner for the photobioreactor 500, such that the algae in the growth medium 105 may be exposed to the light 530 emitted by the light source 215 along the length of the water column 520 in addition to the ambient light that may be incident at the top of the water column 525. The luminescent material 200 may comprise an electrical cord 505 that may be electrically coupled to the power source, such as a conventional AC electrical service 510.

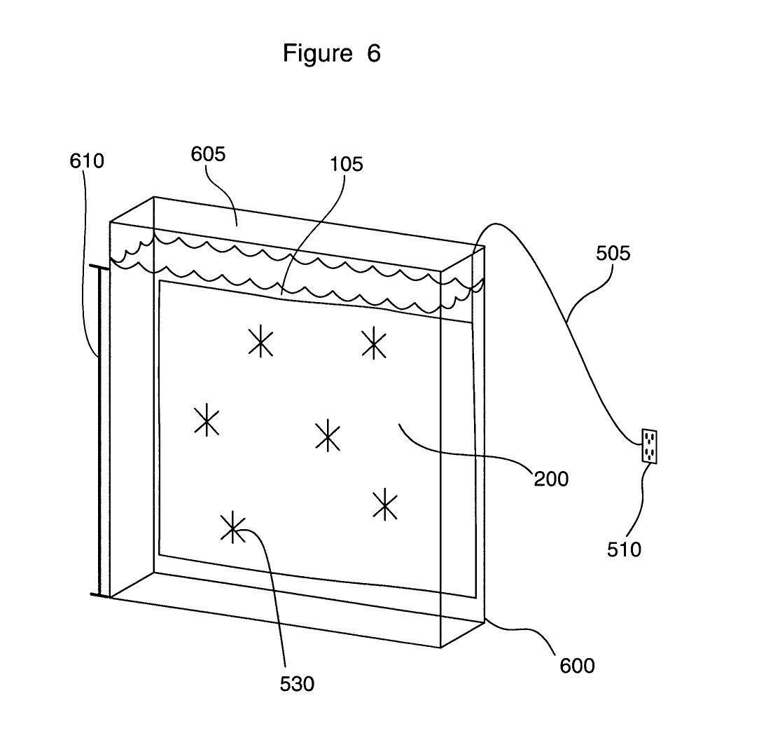

Referring to FIG. 6, in another exemplary embodiment, the luminescent material 200 may be used with a flat panel type photobioreactor 600 for promoting the growth of algae in the growth medium 105. The flat panel type photobioreactor 600 may comprise any photobioreactor having flat walls and short optical paths such that the light 530 emitted by the light source may be transmitted through the width of the flat panel type photobioreactor 600. The luminescent material 200 may be attached to one or more inner surfaces 605 of the flat panel type photobioreactor 600 such that the algae in the growth medium may be exposed to the light 530 emitted by the light source along the length of the water column 610. Alternatively, the luminescent material 200 may be attached to exterior surfaces for transparent walls of the photobioreactor 600 such that the light 530 is transmitted through the walls to the organisms within.

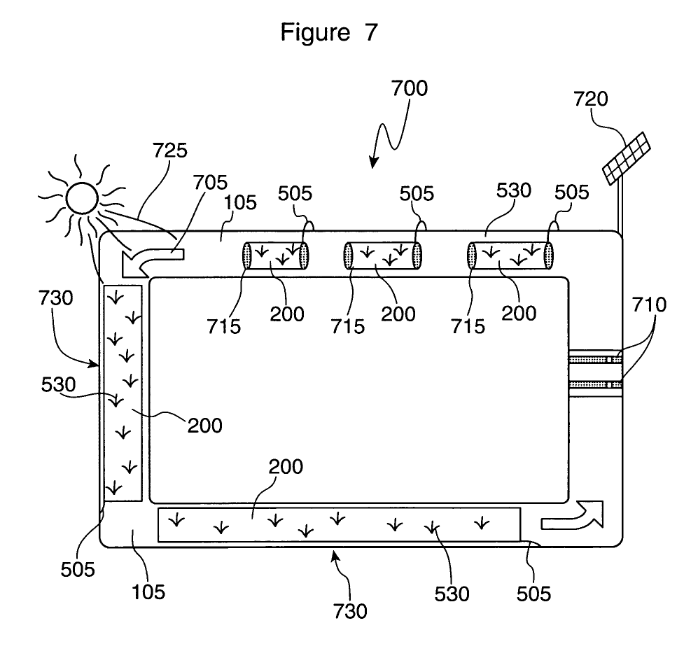

Referring to FIG. 7, in another embodiment according to various aspects of the present invention, the luminescent material 200 may be used in a raceway pond type photobioreactor 700 for promoting the growth of algae in the growth medium 105. The raceway pond type photobioreactor 700 may comprise any natural or man-made pond, and may have a top surface exposed to sunlight 725. In some embodiments, the raceway pond type photobioreactor 700 may comprise paddle wheels 710 that may circulate the growth medium 105 in a particular direction, such as counterclockwise 705. In one embodiment, the luminescent material 200 may lie on the bottom surface of the open-air pond type photobioreactor 700, as shown in area 730. In another embodiment, the luminescent material 200 may be attached to an object 715, such as a length of PVC pipe, and submerged and/or floated in the growth medium 105. In one embodiment, the luminescent material 200 may be electrically coupled to a solar cell 720 or other suitable power source through electrical cord 505.

The luminescent material 200 may form a mat that is constantly exposed to the circulating liquid or partially or completely outside the liquid provided it is kept hydrated. The pond may have any depth including less than about one inch of water that trickles across the mat.

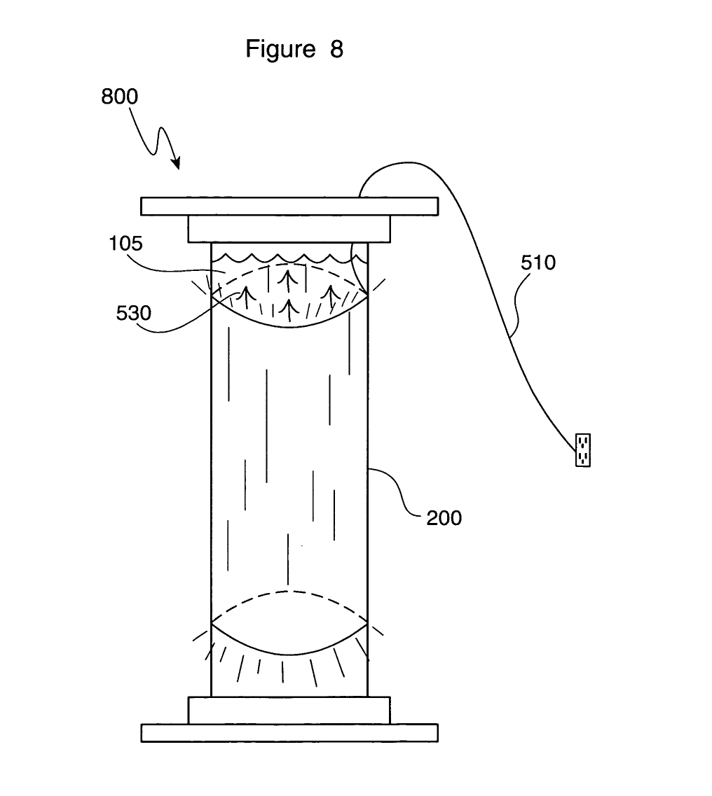

In yet another embodiment, referring to FIG. 8, the luminescent material 200 may be used with a bubble column type photobioreactor 800 for promoting the growth of algae in the growth medium 105. The luminescent material 200 powered by electrical service 510 may be attached to an inner and/or outer surface of the bubble column for exposing the algae within the growth medium 105 to the light 530 emitted by the light source.

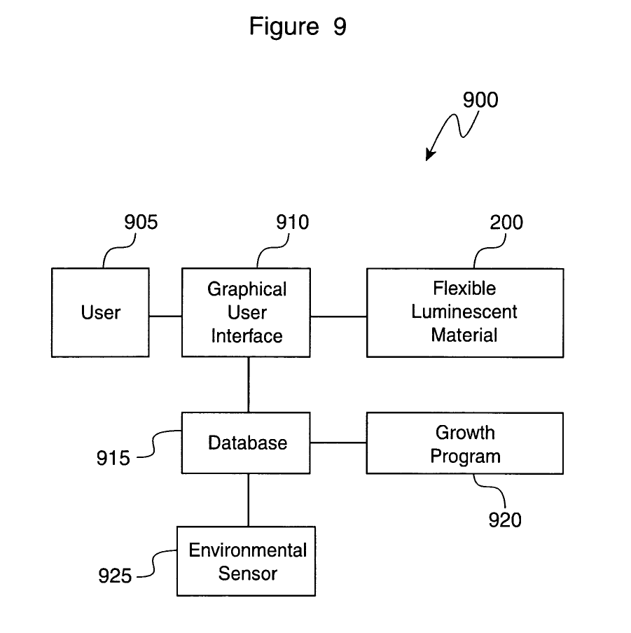

Systems and methods for growing photosynthetic organisms according to various aspects of the present invention may further comprise a control system for controlling the operation of the light source 215. For example, referring to FIG. 9, the luminescent material 200 may further operate in conjunction with a programmable control system 900. The programmable control system 900 may comprise any suitable system for controlling the luminescent material 200. For example, in some embodiments, the programmable control system 900 may comprise a switch, a dimmer, a light timer, and/or a combination thereof.

In an exemplary embodiment of the present invention, the programmable control system 900 may comprise a user interface, such as a graphical user interface 910, a website interface, and/or a computer workstation for access by a user 905. The user interface 910 may comprise any suitable system for communicating, accessing, updating, exchanging information, organizing information, and/or managing information such as by data collection, encryption, acquisition, storage, dissemination, and the like. In one embodiment, the user interface may comprise a website interface, such as for a web server. For example, the web server may comprise a Microsoft® Windows® Internet Information Services (IIS) Web Server.

The graphical user interface 910 may be accessed by a user 905. The user 905 may comprise any individual that may operate the luminescent material 200. For example, the user 905 may be a scientist, photobioreactor engineer, or other support staff that may desire to monitor or modulate the operation of the luminescent material 200. Alternatively, the user 905 may comprise another system, such as control system or computer adapted to control one or more elements of the photobioreactor.

In one embodiment, the graphical user interface 910 may operate in conjunction with a database 915 that may store information entered by the user 905, such as to save the information for the user 905 to access at a later date. The database 915 may also store information related to various growth programs 920 for controlling the light source 215 (LEDs) in the luminescent material 200. In one embodiment, the user 905 may enter the growth program 920 through the user interface 910 to be saved in the database 915. In another embodiment, the growth program 920 may be pre-loaded into the database 915 or generated by the control system 900, for example in conjunction with parameters provided by the user 905. The user 905 may then select and activate the desired growth program 920.

The programmable control system 900 may control any operational aspect of the luminescent material 200. For example, the programmable control system 900 may be adjusted to regulate at least one of an activation or deactivation of the light source 215. The light source 215 may be controlled to provide light at or around a particular monochromatic wavelength, bandwidth of wavelengths, a photoperiod, optical filter, intensity, flashing period (both on and off) and/or other aspect of the light. In one embodiment, the programmable control system 900 may regulate the frequency of the light emitted from the light source 215 to stimulate a photosynthetic rate of the aquatic photosynthetic organism and/or encourage the growth of the aquatic photosynthetic organism when the aquatic photosynthetic organism is exposed to ambient sunlight. The programmable control system 900 may also control the exposure of the aquatic photosynthetic organism to light at a specific time, such as night to reduce dark phase cellular respiration where the aquatic photosynthetic organism is an alga.

In some embodiments, the programmable control system 900 may be adjusted to regulate the light source 215 to elicit any number of pre-selected conditions of the aquatic photosynthetic organism in the growth medium. In one embodiment, the pre-selected condition may comprise any desired density of the aquatic photosynthetic organism in the growth medium. In another embodiment, the pre-selected condition may comprise a desired growth phase of the aquatic photosynthetic organism in the growth medium. In yet another embodiment, the pre-selected condition may comprise the production of a target product from the aquatic photosynthetic organism. For example, the programmable control system 900 may promote the production of photosynthetic pigment as the target product by providing low levels of light in wavelengths efficiently absorbed by the photosynthetic pigment, stimulating the algae to produce more of the photosynthetic pigment.

In various embodiments, the programmable control system 900 may be adjusted to regulate the photoperiod of the light emitted by the light source 215. For example, the photoperiod may be measured on a scale of at least one of hours, minutes, seconds, and milliseconds. In one embodiment, the photoperiod may be adjusted to increase a photosynthetic efficiency (also referred to as a photon absorption efficiency) of the aquatic photosynthetic organism. The photosynthetic efficiency may be the fraction of light energy that is converted into chemical energy by the aquatic photosynthetic organism. In some embodiments, modifying the photoperiod to increase the photosynthetic efficiency of the aquatic photosynthetic organism may have the effect of reducing light-induced photoinhibition, which may damage photosynthetic proteins, such as photosystem proteins in algae.

In an exemplary embodiment of the present invention, the programmable control system 900 may regulate the photoperiod of the light emitted by the light source 215 in terms of a duty-cycle. The duty-cycle may comprise any amount of time the light source 215 is activated to emit light as a fraction of a total amount of a time period, such as minutes, hours, or days. For example, in one embodiment, the light source 215 may be activated to emit light with a 50% duty cycle, such as for 30 seconds each minute. In another embodiment, the light source 215 may be activated to emit light with a non-50% duty cycle. For example, in one embodiment, the light may be emitted in a 25% duty-cycle in which the light is emitted for 15 seconds each minute. The programmable control system 900 may be programmed and/or adjusted to activate the light source 215 at any suitable duty-cycle to increase the photosynthetic efficiency of the aquatic photosynthetic organism.

In some embodiments according to various aspects of the present invention, the programmable control system 900 may activate the light source 215 to emit light at or around a pre-selected wavelength. In one embodiment, the pre-selected wavelength may be determined according to an optimal wavelength absorbance of a photosynthetic pigment (also referred to as a photoreceptor) in the aquatic photosynthetic organism and/or to increase production of the target product. For example, the pre-selected wavelength may optimize the growth of the aquatic photosynthetic organism by providing approximately monochromatic light at a wavelength that is optimally absorbed by the photosynthetic pigment of the aquatic photosynthetic organism. The photosynthetic pigment may comprise any protein produced by the aquatic photosynthetic organism that may capture and absorb light for use in photosynthesis.

In some embodiments, the programmable control system 900 may activate the light source 215 to emit light at a pre-selected wavelength that may be configured to decrease a rate of growth of a competing aquatic photosynthetic organism in the photobioreactor to maintain dominance of the aquatic photosynthetic organism. For example, the pre-selected wavelength may favor growth of the aquatic photosynthetic organism and, at the same time, deny a different wavelength of light needed by the competing aquatic photosynthetic organism to grow optimally where the two organisms have a different complement of photosynthetic pigments. In this case, the aquatic photosynthetic organism may grow optimally to a high density, whereas the competing aquatic photosynthetic organism may be essentially starved for light and may maintain a limited presence in the culture in the photobioreactor.

In an exemplary embodiment of the present invention, the programmable control system 900 may be implemented in any suitable manner and perform any appropriate functions, such as controlling lighting, logging and reporting environmental conditions, and transmitting data. The programmable control system 900 may be configured to monitor individual devices coupled to the luminescent material 200, such as an environmental sensor, and/or may control all of the individual light sources 215 in the luminescent material 200, such as micro-LEDs. The programmable control system 900 may be communicatively linked to the luminescent material 200 and/or associated devices in any suitable manner, such as via coaxial cables, twisted pairs, or networking connections. The programmable control system 900 may communicate through any appropriate medium or connection, such as a wireless connection. Further, the programmable control system 900 may be located in a wall cabinet or control box near the luminescent material 200 that is used with to the photobioreactor, and/or the programmable control system 900 may be located in a remote location, such as a mobile wireless system.

The growth program 920 may comprise any suitable parameters for controlling the light source 215 in the luminescent material 200. For example, the user 905 may enter parameters such as light wavelength, intensity, and/or photoperiod. In one embodiment, the growth program 920 may take into account the level of ambient light, such as sunlight, incident on the photobioreactor when determining the light wavelength and/or intensity of the light to be emitted by the light source 215. For example, the growth program 920 or other program may monitor and/or receive data from an environmental sensor 925, such as a photocell, that may be communicatively linked to the programmable control system 900. In response, the growth program 920 may indicate a change in the light conditions is preferred or the growth program 920 may effect predetermined changes. In another embodiment, the growth program 920 may operate independently from the level and/or characteristics of ambient light incident on the photobioreactor.

The growth program 920 may comprise any appropriate programs for promoting or otherwise affecting the growth of the aquatic photosynthetic organisms. In one embodiment, the growth program 920 may be directed to promoting the accumulation of biomass in terms of grams of aquatic photosynthetic organisms obtained per liter of growth medium 105. For example, the growth program 920 may be configured to activate the output of light from the light source 215 at an intensity that is at the maximum irradiance tolerance (also referred to as photosynthetic photon flux (ppf)) for the species of algae grown in the photobioreactor. For example, the species of algae in the genus Nannochloropsis has a ppf of approximately 2,000 μmol per square meter per second (μmol m⁻² sec⁻¹). Accordingly, the output of light from the light source 215 may be tailored to the ppf needs of specific species of algae in the photobioreactor.

In another embodiment, the intensity of the light emitted from the light source 215 in various portions of the spectrum may be controlled dimmed by the programmable control system 900 to match the desired ppf for various wavelengths. For example, a first portion of the micro-LEDs may be configured to emit light in a blue wavelength, a second portion of the micro-LEDs may be configured to emit light in a red wavelength, and/or a third portion of the micro-LEDs may be configured to emit light in a far red wavelength. In one embodiment, the second portion and the third portion of the micro-LEDs may emit the light at a lower intensity relative to the first portion of micro-LEDs. In that case, the intensity of light in the blue wavelength would be greater than the intensity of light in the red and/or far red wavelengths. In some embodiments, blue wavelength-dominated light may stimulate an increase in cellular mass. Additionally, light in the red wavelengths may stimulate cell division and light in the far red wavelengths may stimulate an increase in a photosynthetic rate of the algae. In one embodiment, the second portion and the third portion of the micro-LEDs may emit the light at an intensity that may be at least 85% of the total intensity of the light emitted by the micro-LEDs.

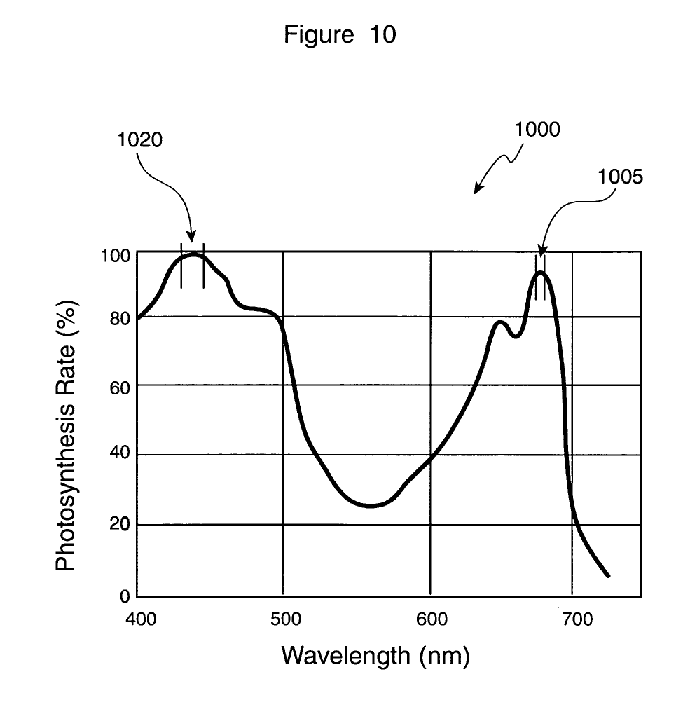

Referring to the photosynthetic spectrum 1000 shown in FIG. 10, the growth program 920 may activate the output of light from the light source in a bandwidth of the light that is substantially centered on a monochromatic wavelength for promoting a maximum rate of photosynthesis. The photosynthesis rate may comprise the rate at which the aquatic photosynthetic organism fixes carbon dioxide in the environment using the energy harvested from light by various photosynthetic pigments. Each species and/or genus of algae may comprise a different complement of photosynthetic pigments, such as chlorophyll proteins. The highest photosynthetic rate may be achieved by providing light to the aquatic photosynthetic organism in the range of approximately 420 nm-460 nm (1020) and/or approximately 650 nm-680 nm (1005), at which various photosynthetic pigments absorb light most efficiently.

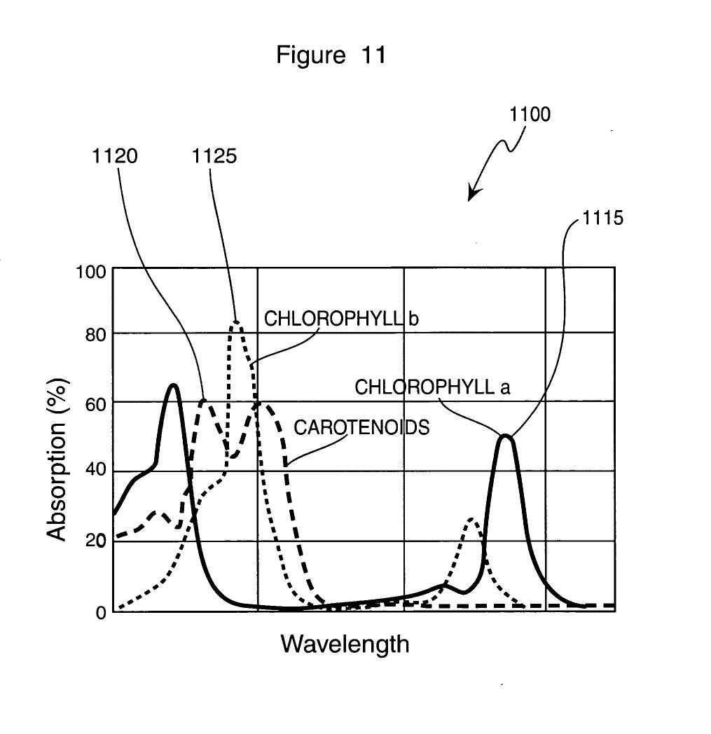

For example, referring to FIG. 11, an exemplary light absorption spectrum 1100 is shown for three photosynthetic pigments. Chlorophyll a absorbs light well at a wavelength of approximately 400 nm-450 nm and at 650 nm-700 nm (1115) and chlorophyll b absorbs light well at approximately 450 nm-500 nm and at 600 nm-650 nm (1125). Carotenoids are photosynthetic pigments that absorb light well at a wavelength of approximately 400 nm-500 nm (1120).

In an exemplary embodiment, the growth program 920 may be configured to optimize the growth rate of the aquatic photosynthetic organism. Where the aquatic photosynthetic organism is an algae, the algae may be exposed to ambient sunlight as a supplementary light source in addition to the light emitted by the light source. In various embodiments, the growth of the algae and/or the accumulation of the target product such as lipids may be accelerated when the algae is exposed to bandwidths of light that may be specific for sunlight at an end of a day and/or a beginning of a day.

For example, the algae may be exposed to a wavelength of light emitted by the light source 215 at an end of a day when an intensity of ambient sunlight is decreasing and/or absent. The wavelengths of light emitted by the light source 215 may be substantially similar to wavelengths of light received from the sun at the end of the day and configured to promote growth of the aquatic photosynthetic organism the next day. In one embodiment, the light source 215 may emit the last bandwidth of light emitted by the sun at sunset, such as 600 nm-800 nm. Exposing the algae to this light for a photoperiod of 5-30 minutes may increase the production of algal growth regulators to boost growth of the algae in the next day.

In another embodiment, the algae may be exposed to a wavelength of light emitted by the light source 215 at a beginning of a day when an intensity of ambient sunlight is increasing and/or absent. The wavelengths of light emitted by the light source 215 may be substantially similar to wavelengths of light received from the sun at the beginning of the day and configured to stimulate a photosynthesis rate of the algae before the sun emits ambient light at peak intensity and/or in other wavelengths. For example, the light source 215 may emit light in a bandwidth characteristic of a sunrise to increase the production of certain growth regulators to stimulate the photosynthesis rate prior to sunrise such that the algae may perform photosynthesis at an optimal and/or peak rate upon sunrise. In some embodiments, these approaches to the growth program 920 may increase the yield of algal biomass produced in the photobioreactor.

In various embodiments, the luminescent material 200 may promote the production of the desired target product in the aquatic photosynthetic organism. The target product may comprise any phytochemical, protein, compound, and/or molecule that the aquatic photosynthetic organism is capable of producing in a biological process. In one embodiment, at least one of the pre-selected wavelengths of light emitted by the light source 215 may stimulate the accumulation of a target product. For example, the algae may be stimulated to promote the accumulation of target products such as proteins, carotenoids, oils, chlorophylls, and phycobilins such as phycoerythrins and phycocyanins by customizing the bandwidth of light emitted by the light source 215. In an exemplary embodiment, at least a portion of the micro-LEDs may emit a lower intensity of light in a wavelength that is optimally absorbed by the phytochemical as compared to the intensity of rest of the light that is provided by the plurality of micro-LEDs. Providing the lower intensity of light in the wavelength optimally absorbed by the phytochemical may stimulate the aquatic photosynthetic organism to increase production of the phytochemical to allow for improved absorption of light at that wavelength that is in low supply.

For example, in one embodiment, algae may be stimulated to accumulate chlorophyll a by reducing the intensity of the light in the approximate range of 400 nm-450 nm and/or 650-700 nm. In another embodiment, the algae may be stimulated to accumulate chlorophyll b by reducing the intensity of the light in the approximate range of 450 nm-500 nm and/or 600-650 nm. In another embodiment, the algae may be stimulated to accumulate phycocyanin by reducing the intensity of the light in the approximate range of 300 nm-400 nm and/or 500-700 nm. In yet another embodiment, the algae may be stimulated to accumulate carotenoids with light in the approximate range of 475 nm to 570 nm. In still another embodiment, the algae may be stimulated to accumulate phycoerythrin by reducing the intensity of the light in the approximate range of 500 nm-600 nm. In response to the reduction of light in this range of wavelength, the algae may increase production of the particular photosynthetic pigment to absorb more light that is in low supply.

In one embodiment, according to various aspects of the present invention, a method for promoting the accumulation of the phytochemical in the photobioreactor may comprise positioning the flexible luminescent material covered with the transparent waterproof material to target the algae in the photobioreactor. The flexible luminescent material may comprise the flexible substrate, the matrix of conductors coupled to the flexible substrate, and the micro-LEDs electrically coupled to the matrix of conductors at a density greater than 25 micro-LEDs per square inch, wherein at least a portion of the micro-LEDs may be configured to emit a lower intensity of light in a wavelength that may be optimally absorbed by the phytochemical as compared to the intensity of rest of the light that is provided by the micro-LEDs. The algae in the photobioreactor may be exposed to the light emitted by the micro-LEDs to stimulate the accumulation of the phytochemical in the algae.

In one embodiment, the luminescent material 200 may promote the accumulation of a lipid. For example, the luminescent material 200 may comprise the first portion of the micro-LEDs configured to emit light in a blue wavelength. The exposure of the algae to light in the blue wavelength may increase the cell mass of the algae and/or may stimulate the accumulation of the lipid, such as biodiesel. In one embodiment, the blue wavelength may be approximately 470 nm.

In an exemplary embodiment, the luminescent material 200 may comprise a second portion of the micro-LEDs configured to emit light in a red wavelength in addition to the first portion of the plurality of micro-LEDs. In one embodiment, the first of the micro-LEDs may emit the light at a higher intensity relative to the second portion of the micro-LEDs. In another embodiment, the luminescent material 200 may comprise a third portion of the micro-LEDs configured to emit light in a far red wavelength in addition to the first portion and the second portion of the micro-LEDs. In one embodiment, the first portion of the micro-LEDs may emit the light at a higher intensity relative to the second portion and the third portion of the micro-LEDs.

In one embodiment, a method for promoting the accumulation of the lipid in the algae within the growth medium in the photobioreactor may comprise positioning the flexible luminescent material covered with the transparent waterproof material to target the algae in the photobioreactor. The flexible luminescent material may comprise the flexible substrate, the matrix of conductors coupled to the flexible substrate, and the micro-LEDs electrically coupled to the matrix of conductors at a density greater than 25 micro-LEDs per square inch, wherein the first portion of the micro-LEDs may be configured to emit light in a blue wavelength. The first portion of the micro-LEDs may be activated and the algae in the photobioreactor may be exposed to the first portion of the micro-LEDs to stimulate the accumulation of the lipid in the algae.

In a further embodiment, the method may comprise the second portion of the micro-LEDs configured to emit light in a red wavelength. The second portion of the micro-LEDs may be activated and the algae in the photobioreactor may be exposed to the light emitted by the second portion of the micro-LEDs, wherein the first portion of the micro-LEDs emits light at a higher intensity relative to the second portion of the micro-LEDs. In still a further embodiment, the method may comprise a third portion of the micro-LEDs configured to emit light in a far red wavelength. The third portion of the micro-LEDs may be activated and the algae in the photobioreactor may be exposed to the light emitted by the third portion of the micro-LEDs, wherein the first portion of the micro-LEDs emits light at a higher intensity relative to the second portion and the third portion of the micro-LEDs.

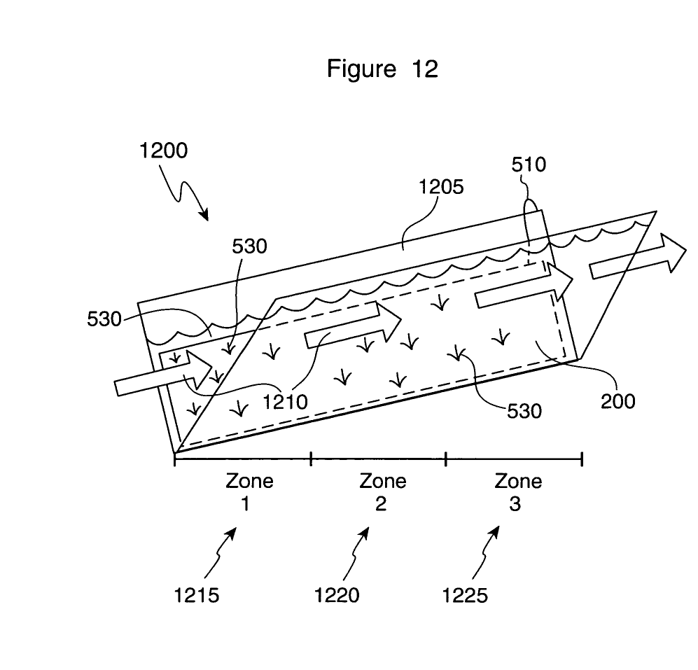

Referring to FIG. 12, in an exemplary embodiment of the present invention, methods of promoting the accumulation of a lipid target product, such as a bio-diesel, in algae may comprise implementing the growth program 920 that may comprise timed sequences of lighting by the light source in an automated continuous photobioreactor 1200. The continuous photobioreactor 1200 may provide for the continuous unidirectional flow 1210 of algae, progressing through different growth phases as the algae passes through the continuous photobioreactor 1200. For example, the algae may progress from an early growth phase in Zone 1 1215, to an early lipid phase in Zone 2 1220, to a lipid phase in Zone 3 1225 that may be ready for harvesting. In one embodiment, the light from the light source may be attached to an inner surface 1205 of the continuous photobioreactor 1200 and may target the flowing algae. The light emitted from the light source as controlled by the programmable control system's 900 growth program 920 may be tailored to promote growth in terms of biomass in the early growth phase of Zone 1 1215 and may promote lipid production in Zone 2 1220 and Zone 3 1225.

Different zones in the present invention and different short term light treatments may be performed by pumping photosynthetic organism through a tube or similar structure where the liquid passes by the luminescent material 200 emitting light 530 which is powered by an electrical service 510. This permits most of the photobioreactor to continue with its general growth method while some of the photosynthetic organism receives specialized treatment.

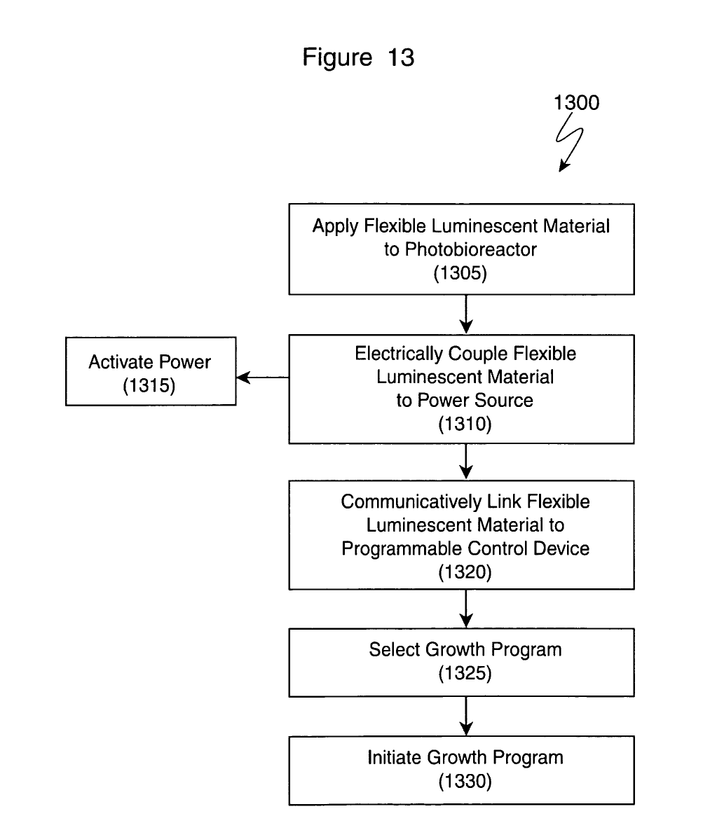

Referring to FIG. 13, an exemplary method of operating a luminescent material according to various aspects of the present invention (1300) may comprise applying the luminescent material to a surface of a photobioreactor, such as by adherence of the luminescent material to an inner surface with suction cups and submerged within the growth medium (1305). The luminescent material may then be electrically coupled to a power source (1310). The power may be activated to flow to the luminescent material, such as by plugging the luminescent material into a wall outlet and/or activation of a power switch (1315). In another embodiment, the luminescent material may be communicatively linked to a programmable control device (1320). A growth program may be selected from the programmable control device's database and/or customized according to a user's needs, such as photoperiod, wavelength, and/or intensity of light (1325). The growth program may then be activated to initiate the emission of light from the light source (LEDs) 215 (1330).

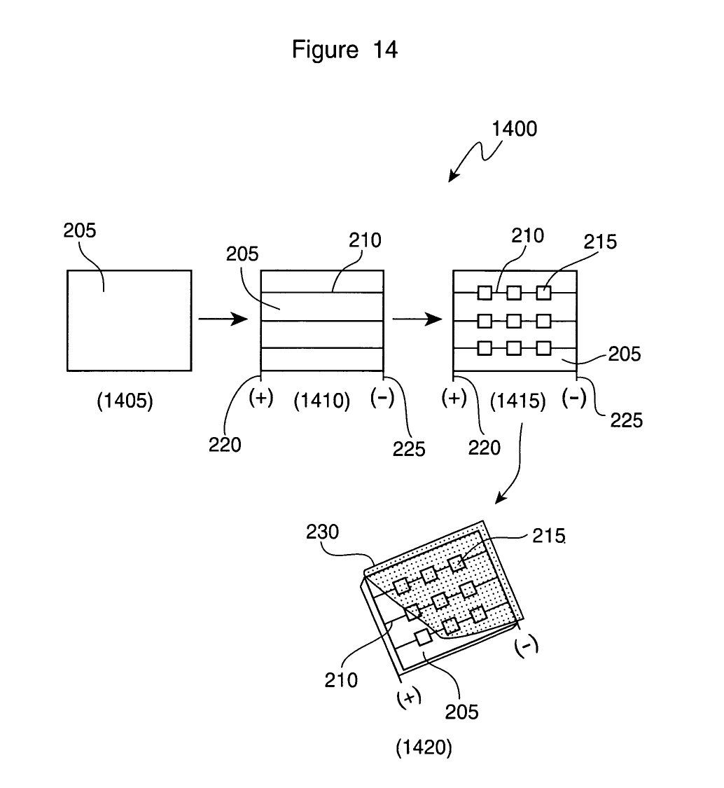

Referring to FIG. 14, an exemplary method of assembling the luminescent material according to various aspects of the present invention may comprise providing a substrate 205, such as a sheet of Mylar (1405). A matrix of conductors 210 may be applied to the surface of the substrate 205, such as by conventional printing of conductive wires onto the substrate 205 and connected to positive electrodes 220 and negative electrodes 225 (1410). Light source 215, such as micro-LEDs, may be electrically coupled to the matrix of conductors 210, such as by conventional printing of the micro-LEDs onto the substrate 205 comprising the matrix of conductors 210 (1415). A protectant 230 may be coupled to the substrate 205 comprising the matrix of conductors 210 and the light source 215, such as by the lamination of a plastic coating (1420).

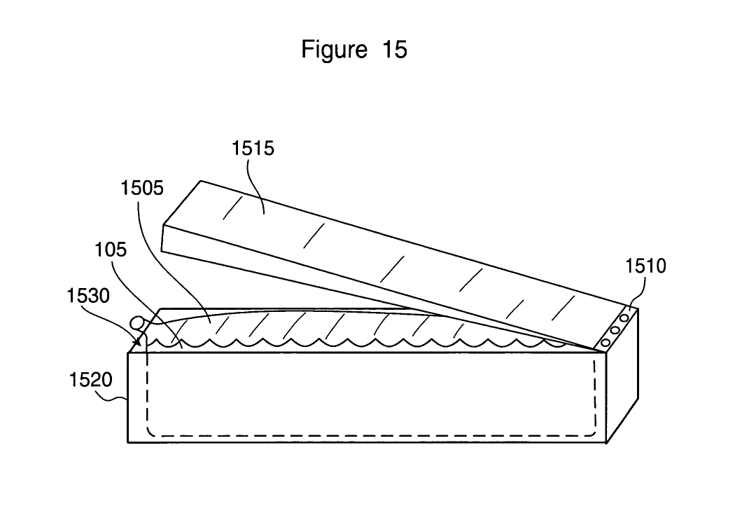

The substrate may be configured in any suitable shape for a particular application or environment. For example, referring to FIG. 15, the luminescent material may comprise at least two substantially rigid substrates 1515, 1520 that may be located adjacent to each other and form a housing having an interior space 1530. The interior space may define or contain a photobioreactor, such as a transparent bag 1505 containing the aquatic photosynthetic organism within the growth medium 105. This “clamshell” or “tanning bed” housing may comprise a fastener 1510 that couples the two substantially rigid substrates together, wherein the fastener 1510 is configured to open and close the two substantially rigid substrates to provide a point of access to the interior space 1530. In one embodiment, the fastener 1510 may comprise a flexible coupling such as a hinge.

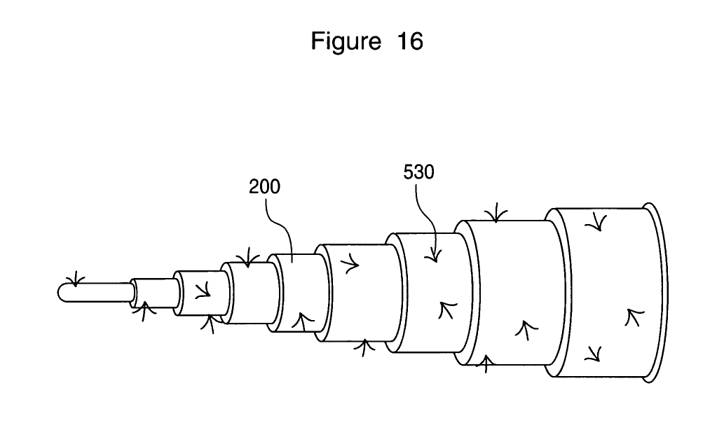

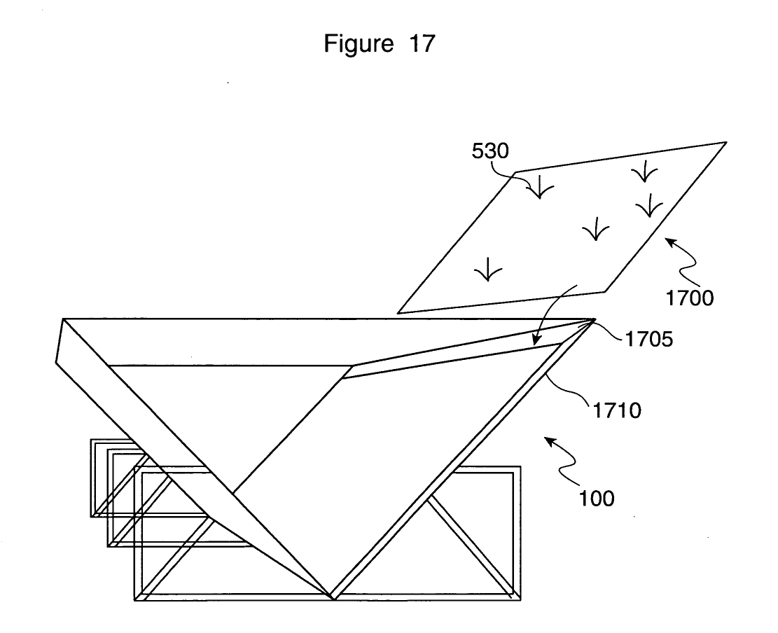

In another embodiment, referring to FIG. 16, the luminescent material 200 capable of emitting light 530 may comprise the substantially rigid substrate configured as a tube that may be extended through a length of the photobioreactor, such as by telescoping. In another embodiment, referring to FIG. 17, the luminescent material capable of emitting light 530 may comprise the substantially rigid substrate configured as a sheet 1700 that may be inserted into a slot 1705 in a wall 1710 of the photobioreactor 100.

In the foregoing description, the invention has been described with reference to specific exemplary embodiments. Various modifications and changes may be made, however, without departing from the scope of the present invention as set forth. The description and figures are to be regarded in an illustrative manner rather than a restrictive one, and all such modifications are intended to be included within the scope of the present invention. Accordingly, the scope of the invention should be determined by the generic embodiments described and their legal equivalents rather than by merely the specific examples described above. For example, the steps recited in any method or process embodiment may be executed in any appropriate order and are not limited to the explicit order presented in the specific examples. Additionally, the components and/or elements recited in any system embodiment may be combined in a variety of permutations to produce substantially the same result as the present invention and are accordingly not limited to the specific configuration recited in the specific examples.

Benefits, other advantages, and solutions to problems have been described above with regard to particular embodiments. Any benefit, advantage, solution to problems, or any element that may cause any particular benefit, advantage, or solution to occur or to become more pronounced, however, is not to be construed as a critical, required, or essential feature or component.

The terms “comprises,” “comprising,” or any variation thereof, are intended to reference a non-exclusive inclusion, such that a process, method, article, composition, system, or apparatus that comprises a list of elements does not include only those elements recited, but may also include other elements not expressly listed or inherent to such process, method, article, composition, system, or apparatus. Other combinations and/or modifications of the above-described structures, arrangements, applications, proportions, elements, materials, or components used in the practice of the present invention, in addition to those not specifically recited, may be varied or otherwise particularly adapted to specific environments, manufacturing specifications, design parameters or other operating requirements without departing from the general principles of the same.

The present invention has been described above with reference to an exemplary embodiment. However, changes and modifications may be made to the exemplary embodiment without departing from the scope of the present invention. These and other changes or modifications are intended to be included within the scope of the present invention.

We claim:

1. A luminescent material for growing a photosynthetic organism within a culture medium in a photobioreactor comprising;
a substrate,
a plurality of luminescent semiconductor devices disposed on the substrate at a density of greater than 10 luminescent semiconductor devices per square inch, electrical connections disposed on the substrate connecting to the plurality of luminescent semiconductor devices; and
a waterproof covering over the luminescent semiconductor devices and the electrical connections,
wherein the luminescent material is positionable inside the culture medium in a photobioreactor and is capable of exposing the photosynthetic organism within the culture medium to light emitted by the luminescent material.

2. The luminescent material of claim 1 wherein the luminescent semiconductor is a micro-scale light emitting diode.

3. The luminescent material of claim 1, further comprising a diffuser or phosphor mounted on top of the luminescent semiconductor device.

4. The luminescent material of claim 1, wherein the luminescent material is flexible.

5. The luminescent material of claim 1, wherein the waterproof covering is bound to the substrate.

6. The luminescent material of claim 1, further comprising an anti-biofouling agent in the waterproof covering.

7. A method for culturing a photosynthetic organism in a culture medium comprising;
submerging the luminescent material of claim 1 into a liquid to become a culture medium containing the photosynthetic organism, and
powering the luminescent semiconductor device,
wherein the luminescent material emits light to the photosynthetic organism in the culture medium.

8. A system for growing an photosynthetic organism in a culture medium comprising;
photobioreactor and
a luminescent material comprising;
a substrate,
a plurality of luminescent semiconductor devices disposed on the substrate at a density of greater than 10 luminescent semiconductor devices per square inch, and
electrical connections disposed on the substrate connecting to the plurality of luminescent semiconductor devices;
wherein the luminescent material is capable of exposing the photosynthetic organism within the culture medium to light emitted by the luminescent material.

9. The system claim 8 wherein the luminescent semiconductor is a micro-scale light emitting diode.

10. The system of claim 8, wherein the luminescent material is flexible.

11. The system of claim 8, wherein the luminescent material is located inside the photobioreactor.

12. The system of claim 8 wherein the luminescent material is located within a wall of the photobioreactor.

13. The system of claim 8 wherein the density of luminescent semiconductor devices is greater than 100 per square inch.

14. The system of claim 8 wherein the luminescent material further comprises a waterproof covering.

15. The system of claim 8 wherein the luminescent material further comprises a divider within the photobioreactor.

16. The system of claim 8 wherein the luminescent material further comprises a cylinder.

17. The system of claim 16 wherein the cylinder is a pipe through which the photosynthetic organism within the culture medium travels.

18. A method for culturing a photosynthetic organism in a culture medium comprising;
submerging the luminescent material of claim 8 into a liquid to become a culture medium containing the photosynthetic organism, and
powering the luminescent semiconductor device,
wherein the luminescent material emits light to the photosynthetic organism within the culture medium.

19. The system of claim 8, further comprising a programmable control system communicatively linked to the luminescent material, wherein the programmable control system is configured to regulate at least one of an activation and deactivation of each luminescent semiconductor device, a photoperiod, and an intensity of the light emitted by the luminescent semiconductor device.

20. The system of claim 19, wherein the programmable control system is further configured to regulate the luminescent semiconductor devices to achieve a pre-selected condition of the photosynthetic organism in the growth medium, the pre-selected condition comprising at least one of a pre-selected density of the aquatic photosynthetic organism in the growth medium, a growth phase of the aquatic photosynthetic organism, and the accumulation of a target product produced by the aquatic photosynthetic organism.

21. The system of claim 8, wherein each of luminescent semiconductor devices emits one bandwidth of light substantially centered around a monochromatic wavelength, and wherein different luminescent semiconductor devices emit different bandwidths centered around different monochromatic wavelengths.

22. The system of claim 21 wherein each bandwidth of light has a different effect on the photosynthetic organism.

23. The system of claim 8, wherein the luminescent material emits either a light according to an optimal wavelength absorbance efficiency of a photosynthetic pigment in the photosynthetic organism or a light to elicit a pre-selected condition of the photosynthetic organism.

24. The system of claim 23, wherein the optimal wavelength enhances production of a target product.

25. The system of claim 8, wherein the luminescent material emits an optimal wavelength for killing, inactivating or degrading a contaminant in the culture medium either with or without a photocatalyst.

26. A method for growing a photosynthetic organism in a culture medium comprising,
adding a photosynthetic organism and culture medium to the photobioreactor in the system of claim 19, and
activating the programmable control system to control the culture
wherein the luminescent material is activated and the photosynthetic organism is cultured.

27. A system for growing an photosynthetic organism in a culture medium comprising;
photobioreactor and
a luminescent material comprising;
a substrate,
a plurality of optical fibers having a light emitting region disposed on the substrate at a density of greater than 10 light emitting regions per square inch, and
a light source providing light to the optical fibers;
wherein the luminescent material is capable of exposing the photosynthetic organism within the culture medium to light emitted by the optical fibers.

28. The system of claim 27 wherein a bundle of greater than 100 optical fibers is present.

29. The system of claim 27 wherein the density is greater than 100 light emitting regions per square inch.

30. The luminescent material of claim 1, wherein at least two different luminescent semiconductor devices are present with each emitting a different band of light around a different wavelength.
